(19) United States
(12) Patent Application Publication
(10) Pub. No.: US 2010/0010451 A1
(43) Pub. Date: Jan. 14, 2010

(54) Huber Needle Assembly and Method of Use

(76) Inventors: Avtar Singh Kashmirian, Australian Capital Territory (AU); Craig Douglas Wilson, New South Wales (AU); David Evan Bassetti, New South Wales (AU); Chris Whelan, New South Wales (AU)

Correspondence Address: JOHN ALEXANDER GALBREATH, 2516 CHESTNUT WOODS CT, REISTERSTOWN, MD, 21136, US

(21) Appl. No.: 12/302,783

(22) PCT Filed: May 27, 2007
(86) PCT No.: PCT/AU2007/000732

§ 371 Date: Feb. 24, 2009

(30) Foreign Application Priority Data
May 26, 2006 (AU) 2006902842

(51) Int. Cl. A61M 5/32

(52) U.S. Cl. 604/192

(57) ABSTRACT

A Huber needle assembly (10) has a base (12) with hinged guide members (20) about a needle aperture (28). A needle assembly (14) has a Huber needle (40) mounted between two hinged wings (54). The guide members (20 ) are movable between a parallel arrangement in which the needle may move vertically between the guide members (20) and a horizontal arrangement in which the needle (40) is locked in the in use position.

### TECHNICAL FIELD

This invention relates to Huber needle assemblies of the right angle type in which a fluid delivery or extraction tube is arranged substantially at right angles to the needle point. This is normally achieved by employing a metal needle having a right angle bend. The invention is also concerned with methods of using Huber needle assemblies.

### BACKGROUND OF THE INVENTION

Huber needle assemblies are commonly used to access subcutaneously implanted medication ports that may be connected to patients' blood vessels or organs for the delivery of a series of liquid medications over an extended period of time for the purpose of chemotherapy, pain management, hormone delivery, microbial control or the like. In some cases, the ports act as reservoirs of therapeutic drugs that are replenished from time to time liquid using a Huber needle. The implanted ports normally have septums of silicone plastic that lie a little below the skin and can be penetrated by a Huber needle that is inserted at right angles to the skin. It is usual therefore for the Huber needle to have a right angle bend so that the butt of the needle extends substantially parallel to the skin. A trailing tube is normally connected to the needle butt and is used to supply the medication to the needle. In rare cases Huber needle assemblies can be used to extract liquid from the patient, whether via a subcutaneous port or otherwise. The use of a right angle needle allows the assembly to have a low profile so that it is easily taped in place and is unlikely to catch on clothing or bed linen. The danger of needle-stick injury from Huber needles is well recognized and most Huber needles currently on the market are provided with needle-point guards that are activated in one way or another upon withdrawal of the needle from the port. The term ‘Huber needle assembly’ is commonly applied to the needle (whether bent or not), its trailing tube, needle guard and any finger grips or the like used to assist needle insertion and withdrawal into a subcutaneous port.

A particular problem with Huber needles is ‘rebound needle-stick’ because of the considerable force required to pull the needle point back out of the port septum. Rebound needle-stick commonly occurs because the user presses the fingers of the left hand on the skin of the patient close to the port before pulling the needle out of the port with the right hand. This is done to reduce patient discomfort and the likelihood of moving the port under the skin. However, as the needle is suddenly released by the septum, the tendency is for the user to try to inhibit the sudden movement with the result that the right hand rebounds and drives the needle point into the top of the fingers of the left hand before any needle guard can be activated.

### BACKGROUND ART

U.S. Pat. No. 6,921,388 to Swenson discloses a Huber needle assembly in which the bent butt of the needle is molded in to a needle holder and a guard is pivoted from the hub so that can be swung downwards to cover the needle point after withdrawal. Unfortunately, this hardly mitigates the danger of rebound needle-stick because such injury is highly likely to occur before the guard can be swung into place. US patent applications 2004/0049159 and 2004/0072716 by Barrus et al disclose a Huber needle assembly where the needle hub is connected by a toggle-like joint (that doubles as a needle guard) to a pad that rests on the skin of the patient. Withdrawal of the needle and activation of the guard therefore requires the user to hold the pad with the fingers of the left hand very close to the point of the needle before it is fully withdrawn into the guard. Withdrawal of the needle without pressing down on the pad results in a fully exposed needle being withdrawn (with the danger of rebound needle-stick) and the need to then manipulate the assembly to effect guarding of the exposed needle (with the additional danger of ‘normal’ needle-stick injury). A similar danger is evident in the simpler guarded Huber needle assembly of U.S. Pat. No. 5,951,522 to Rosato where the guard is a sort of lazy tongs that can be pushed down over the needle after withdrawal. It might be noted that many other older designs of Huber needle assemblies—for example, that disclosed in U.S. Pat. No. 4,627,843—require the attachment or activation of a needle guard after withdrawal and, therefore, after exposing the user to the danger of rebound needle-stick.

A guarded needle assembly is disclosed in U.S. Pat. No. 6,261,259 to Bell that largely avoids the problem of rebound needle-stick injury by encasing needle hub in a housing and withdrawing it into the housing with the needle. If this assembly is used as intended, the housing is pressed down around the puncture site as the needle is being withdrawn, but it is prone to inadvertent misuse by simply pulling on the housing to withdraw the needle, thereby dangerously exposing the user to rebound needle-stick. Also, being bulky, the housing does not lie flat on the patient's skin and is in danger of catching on clothing or bed linen.

### DISCLOSURE OF THE INVENTION

In one form, the Huber needle assembly of the present invention basically comprises a base sub-assembly and a needle sub-assembly that is guided by the base sub-assembly during insertion and withdrawal of the needle point. The base sub-assembly preferably has a substantially horizontal foot adapted to rest, directly or indirectly, on the patient's skin (which is assumed for convenience to be horizontal) and guide means adapted for guiding the needle sub-assembly. The guide means can be arranged for movement between a substantially vertical position in which it can guide the needle during insertion and/or withdrawal and a substantially horizontal position in which it lies out of the way (preferably on or near the foot) after the needle has been inserted. The needle sub-assembly preferably includes a needle hub formed on the needle and a finger-grip attached to the hub for use in manipulating the needle point during insertion and withdrawal. Preferably, the finger-grip is also moveable between a substantially vertical position in which it can be used to insert and/or remove the needle and a substantially horizontal position (preferably on or near the foot) in which it out of the way after the needle has been inserted. Preferably, the needle sub-assembly slidingly engages the guide means during insertion and withdrawal and the guide means preferably acts as a needle guard before and/or after withdrawal of the needle, when the guide is substantially vertical. In that case, it is also preferable that some form of lock is provided between the needle sub-assembly and the base sub-assembly so that, after withdrawal, the needle point cannot be re-extended or re-exposed.

The guide means may include a pair of opposed elongate guide members each of which can be hingedly attached to the foot base sub-assembly so that, when vertical, the two members are in parallel spaced-apart relation and, when substantially horizontal, they lie on or near the substantially horizontal portion of the base. For example, the guide members may be swung or folded outwards and downwards in opposite directions when being moved from their respective vertical positions to their respective horizontal positions. Conveniently, the guide members and the foot of the base sub-assembly can be formed as a single piece plastic molding in which the connection between the bottom of each guide member and the foot is an integral hinge.

As is conventional, the needle may be formed from a hollow metal tube that is bent at right angles so that the pointed portion is normally vertical and the butt portion is normally horizontal. The needle hub is preferably molded from plastic around the butt portion of the metal needle and the finger-grip is preferably formed as two wing-like extensions (also called ‘wings’ herein) extending from opposite sides of the hub, the wings being attached to the hub by integral plastic hinges so that they can be swung upwards together to the vertical position to form the finger grip and also swung downwards and away from one another out of the way to substantially horizontal positions on or near the foot of the base sub-assembly. A slot may be formed in each wing near the hub to take a respective one of the guide members so that, when the guides are arranged vertically and entered into their respective slots, the needle sub-assembly (comprising the needle, hub and wings) can slide up and down on the guide members to allow the insertion and withdrawal of the needle point. Thus, after the needle has been inserted, the guide member and wing member on each side of the assembly can be swung downwards together to a substantially horizontal position so that the wing member lies between the foot and its respective guide member.

In other arrangements, the foot of the base sub-assembly can be elongated or slotted so that the wings and/or guide members can rest, directly or indirectly, on the skin of the patient rather than on the foot itself.

As noted above, when the needle sub-assembly is raised, the needle point is preferably located between the guide members of the base sub-assembly which then serve as guards that prevent finger contact with the needle point. As also noted, it is preferable that some form of locking means is formed between the needle sub-assembly and the base sub-assembly so that, once fully withdrawn upwardly, the needle sub-assembly cannot be again pushed down to re-expose the needle point. This is conveniently achieved by arranging the lock means to operate between the wings and the guide members.

The Huber needle assembly of this invention may be shipped with the guide members and the wings clipped together and arranged in the vertical position with the pointed portion of the needle fully exposed below the foot of the base, the needle point being protected by a removable sheath. The Huber needle assembly can be held by gripping the wings and guide members between the fingers and thumb of the right hand so that the protective needle sheath can be removed using the left hand. With the wings and guide firmly held between the fingers and thumb of the right hand, the needle point is positioned over the insertion point so that the guide members are substantially vertical (ie, at right angles to the skin of the patient at the insertion point) and the needle is firmly inserted into the implanted port through the skin by pushing the entire assembly downwards until the foot of the base sub-assembly rests on the skin. The wings and guide members are then folded outwards and downwards away from one another to their horizontal positions and then taped onto the skin of the patient. If desired, however, the foot may be taped down separately from the guide members and wings.

After sufficient medication has been delivered, the tape applied over the wings and guide members is removed and the wings and guide members are swung upwardly to their original vertical positions using one hand while the fingers of the other hand steady the foot of the base sub-assembly against movement relative to the skin of the patient. Any further tape applied to the foot is then also removed while keeping it steady. The user then presses the foot of the base sub-assembly firmly onto the patient while pulling upward on the wings of the needle sub-assembly so as to slide them up the guide members and so withdraw the pointed portion of the needle upwards between the guide members until the locking means is activated (preferably indicated by an audible click or by feel), Whereupon, the left hand is withdrawn and entire Huber needle assembly is removed from the patient for disposal.

In one optional variant or refinement, the wings are slotted or channeled to take the guide members so that, when the wings are pressed together to effect withdrawal of the needle, their peripheries contact one another rather than pressing the guide members together onto the needle hub. This ensures that (i) the wings and guides cannot be angularly misaligned during withdrawal of the needle so that its point might be exposed from the sides of the guides, (ii) the relative sliding movement between the wings and the guides members is minimally affected by finger pressure on the wings, and (iii) and the sliding movement of the needle hub between the guide members is not unduly inhibited by friction with the guide members. Furthermore, this arrangement allows ratchet-like catches to be incorporated along the edges of the channels in the wings for engagement with complementary catches on the edges of the guide members to prevent the return of a withdrawn needle to the exposed position. Such catches can thus form part of the locking means by which return of the needle is prevented or at least inhibited.

In another optional variant, one or both of the guide members may be provided with a longitudinal (normally vertical) rib that engages a slot in the corresponding wing so that relative arcuate movement of the wing relative to the guide is prevented or substantially restrained. Again, the use of such a rib allows the use of locking means operable between the wing and the rib for inhibiting return of the needle to the exposed position.

In another broad form the invention provides a needle assembly including:
a base sub-assembly having:
   a foot portion having a generally horizontal base surface adapted to rest, directly or indirectly, on a patient's skin and having a passageway through which a needle may pass into the patient, and
   at least one guide member mounted on the foot portion and movable between a generally vertical position and a generally horizontal position, and
   a needle sub-assembly, including a needle having a point and a hub formed on the needle, slidably mounted on the at least one guide member
   wherein, when the at least one guide member is in the generally vertical position, the needle sub-assembly may slide along the at least one guide member, for insertion and/or withdrawal of the needle, between an extended position, in which the needle extends through the passageway and a retracted position in which the needle point is withdrawn away from the base surface.

In the withdrawn position the point of the needle may be located within the passageway and/or at least partially shrouded by the at least one guide means.

The at least one guide member may act as a needle guard before and/or after withdrawal of the needle, when the at least one guide member is substantially vertical.

The needle assembly preferably includes locking means between the needle sub-assembly and the base sub-assembly so that, after withdrawal to the withdrawn position, the needle point cannot be re-extended or re-exposed.

The needle assembly preferably includes retaining means to prevent the needle sub-assembly being separated from the base sub-assembly.

Preferably, when assembled, the at least one guide member is movable between the horizontal and vertical positions only when the needle sub-assembly is in the extended position.

Preferably at least part of the at least one guide member extends through the needle sub assembly.

The needle sub-assembly may include at least one finger grip attached to the hub for use in manipulating the needle point during insertion and withdrawal.

The at least one finger grip is preferably movable between a generally vertical position in which it can be used to insert and/or remove the needle and a generally horizontal position in which it lies out of the way after the needle has been inserted. The at least one finger grip may be hinged to the hub. The at least one guide member may extend between the hub and the at least one finger grip.

The or each at least one finger grip may be mounted to the hub by two strip-like hinges, the two strip like hinges defining a slot therebetween through which a corresponding guide member may extend. Preferably the needle sub-assembly includes two finger grips extending from either side of the hub.

The at least one guide member may be hingedly mounted on the foot portion.

Preferably there are two guide members and more preferably the two guide members extend between two finger grips. Preferably the hub is located between the two guide members.

The at least one guide member, in the generally horizontal position, may overlie at least part of the needle sub assembly and prevents or resists movement upwards movement of the needle sub-assembly toward the withdrawn position.

The needle assembly may include at least one guide slot on one of the guide means and the needle sub assembly and at least one corresponding guide tab on the other of the guide means and the needle sub assembly that engages in the guide slot.

The guide slot may have a locking detent and/or a protrusion that, once the needle sub assembly has been moved to the withdrawn position, prevents movement of guide tab and the needle sub assembly past the detent toward the extended position.

The needle sub-assembly may include at least one first resilient leg that, once the needle sub assembly has been moved to the withdrawn position, overlies at least one free end of the at least one guide member and prevents or limits movement of the needle sub assembly toward the extended position.

There may be at least second one resilient leg on the at least one guide member that engages a part of the needle sub-assembly to prevent the needle sub-assembly being separated from the base sub-assembly. Preferably the at least one second resilient leg engages the needle hub.

Preferably, in the generally horizontal position, the at least one guide member extends away from the passageway after the needle has been inserted.

As indicated above, the invention also comprises a method of the using a Huber needle assembly of the general type and in the general manner just indicated. More specifically, the needle assembly is preferably one in which a needle sub-assembly is slidingly mounted on a base sub-assembly for insertion removal of the needle point, the base sub-assembly having a foot adapted to rest on a patient's skin at the site of insertion and having guide means for the needle sub-assembly, and in which the needle sub-assembly has a finger-grip by which it can be moved relative to the base sub-assembly. The method preferably includes the steps of:
using the finger-grip to insert the needle point vertically until the foot substantially rests on the skin of the patient,
folding the guide means and the finger grip to substantially horizontal positions so that they can be taped in substantially flat positions,
after using the inserted needle to infuse or withdraw fluid, folding the guide means and the finger grip substantially vertical,
holding the foot against movement relative to the skin of the patient with one hand, and
withdrawing the needle from the patient by pressing on the foot with one hand and pulling upward on the finger grip with the other hand so as to slide the finger grip and the needle hub upwards and slidingly relative to the guide means.

It will be noted that rebound needle-stick cannot occur in the procedure described because the needle assembly is held firmly on the skin of the patient until the needle is fully withdrawn. Furthermore, since the guide members and the wings can be quite slim and can fit into recesses on the horizontal portion of the base sub-assembly, the needle assembly will lie flat on the skin of the patient and will not be prone to catching on clothing or bed linen. It can also be taped down in a simple and effective manner. In addition, since the wings of the needle sub-assembly can be made as long and as wide as desired, a good gripping surface can be formed for sure and convenient movement of the needle sub-assembly during insertion and withdrawal of the needle.

### BRIEF DESCRIPTION OF THE DRAWINGS

Having broadly portrayed the nature of the present invention, two examples will be described below with reference to the accompanying drawings, in which:

FIG. 1 is a perspective exploded view of the first example of a Huber needle assembly according to the invention.

FIG. 2 is a perspective view of the Huber needle assembly of FIG. 1, as supplied and ready for use (after having been removed from its packaging, which is not shown).

FIG. 3 is a side view of the Huber needle assembly of FIG. 1, ready for use.

FIG. 4 is a perspective sectional view the Huber needle assembly of FIG. 1 ready for use.

FIG. 5 is a perspective view of the Huber needle assembly of FIG. 1 arranged as it would be when in use.

FIG. 6 is a perspective view of the Huber needle assembly of FIG. 1 arranged as it would be after the needle has been fully withdrawn.

FIG. 7 is a perspective sectional view the Huber needle assembly of FIG. 1 arranged as it would be after the needle has been fully withdrawn.

FIG. 8 is a perspective exploded view of the second example of a Huber needle assembly according to the invention.

FIG. 9 is a reverse perspective exploded view of the second example.

FIG. 10 is a perspective view of the second example after assembly in the horizontal position.

FIG. 11 is a perspective view of the second example, as supplied and ready for use (after having been removed from its packaging, which is not shown).

FIG. 12 is a reverse perspective view of the second example, as supplied and ready for use (after having been removed from its packaging, which is not shown).

FIG. 13 is a perspective sectional view the second example ready for use.

FIG. 14 is a perspective view from below of the second example arranged as it would be after the needle has been fully withdrawn.

FIG. 15 is a perspective view from above of the second example arranged as it would be after the needle has been fully withdrawn.

FIG. 16 is a perspective sectional view the second example arranged as it would be after the needle has been fully withdrawn.

### DETAILED DESCRIPTION OF EXAMPLES OF THE INVENTION

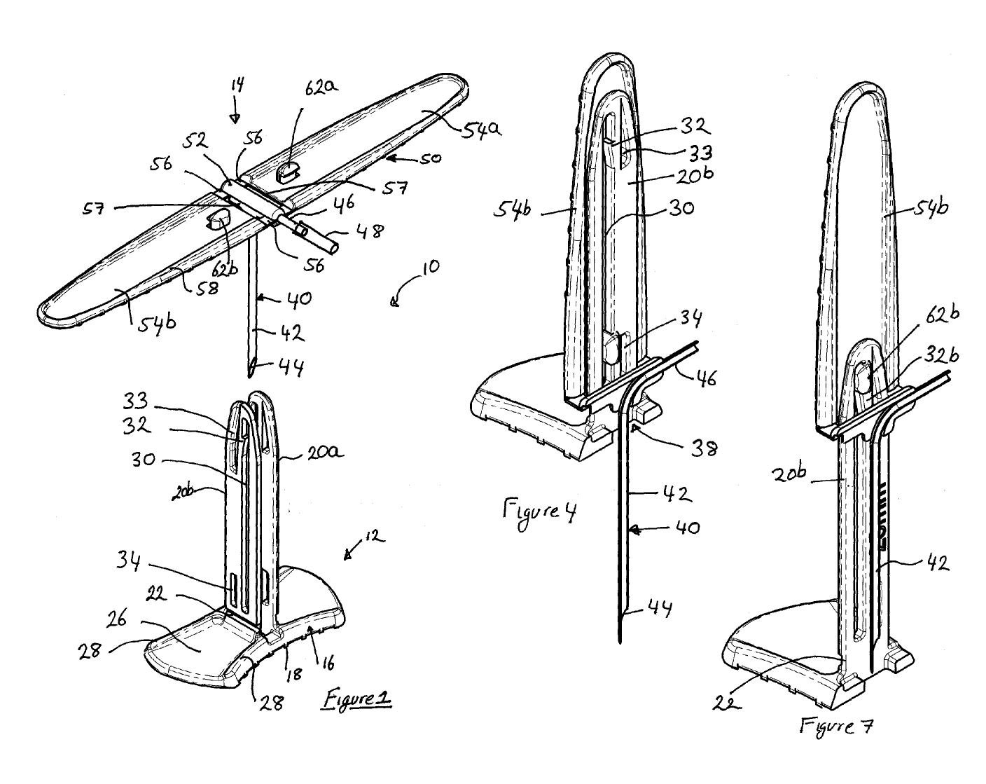

The Huber needle assembly of a first example, shown in FIGS. 1 to 7, is generally indicated at 10 and its component base and needle sub-assemblies at 12 and 14 respectively.

The base sub-assembly 12 is conveniently injection-molded in one-piece from a plastic such as PVC or polypropylene so as to have an elongate foot 16 with a ribbed or patterned bottom face 18 and a pair of opposed strip-like guide members 20a and 20b that are each attached by a narrow integral strip-hinge 22 to foot 16 near its center. In use the base sub-assembly 12 is positioned above the injection site and a layer of foam or other padding is sandwiched between the bottom face 18 and the patient's skin. Guide members 20a and 20b can be swung outwards and downwards about their respective hinges 22 to lie on or near the upper surface 26 of foot 16 which is channeled or grooved for the purpose, having raised sides 28. A long slot 30 with closed ends is formed off-center in each guide member 20a and 20b and extends for almost the full length of the guide member, the outer or upper end of each slot 30 being shaped to form a ratchet-like detent 32. A much shorter slot 33 is formed in each member 20a and 20b opposite detent 32 to facilitate lateral deflection of the detent 32 within the plane of the respective member. A similar short slot 34 is formed near the bottom of each guide member 20a and 20b to allow lateral deflection of the bottom end or contour of long slot 30. Finally a central hole 38 is formed in foot 16 between the strip hinges 22 of guide members 20a and 20b through which the pointed portion of a Huber needle can extend.

The needle sub-assembly 14 includes a bent hollow steel Huber needle 40 having a vertical portion 42 formed with a point 44 and a horizontal butt portion 46 to the end of which a flexible tube 48 is attached for conveying fluid to or from the lumen of needle 40. Sub-assembly 14 also includes a one-piece plastic molding 50 having a central cylindrical hub 52 that is molded onto butt portion 46 of needle 40 and a pair of lateral wings 54a and 54b attached to hub 52 by integral strip-hinges 56. An elongate slot 57 is formed in the center of each strip-hinge 56 and, in fact, reduces the strip-hinge to a two peripheral or end portions. An upstanding peripheral rim 58 is formed around each wing 54a and 54b. Hook-like catches 62a and 62b are formed near the bottom or inner ends of wings 54a and 54b so as to be upstanding from upper or inner faces of wings 54a and 54b, respectively.

Base sub-assembly 12 and needle sub-assembly 14 are assembled by the following steps:
Moving the two sub-assemblies toward one another from the positions shown in FIG. 1 so that vertical portion 42 of needle 40 is centrally aligned between guide members 20a and 20b,
Entering the upper ends of guide members 20a and 20b into respective slots 57 in hinges 56 of wings 54a and 54b,
Continuing this movement until, first, needle point 44 enters central hole 38 in foot 16, and second, wings 54a and 54b contact foot 16,
Swinging wings 54a and 54b about hinges 56 upwardly toward guide members 20a and 20b, respectively, until hook-like catches 62a and 62b abut the outer faces of guide members 20a and 20b,
Pressing guide member 20a and wing 54a firmly together to force catch 62a through long slot 30 in guide member 20a, snap-like entry of catch 62a being facilitated by the flexibility of the lower portion of slot 30 contributed by parallel slot 34 in member 20a, and
Pressing guide member 20b and wing 54b firmly together to force catch 62b through long slot 30 in guide member 20b, snap-like entry of catch 62b being facilitated by the flexibility of the lower portion of slot 30 contributed by parallel slot 34 in member 20b.

It is to be noted that snapping of catch 62a into slot 30 lock in guide 20a prevents these two components from being separated again while allowing upward sliding movement of wing 54a with respect to guide 20a (and foot 16), which movement is limited by the abutment of catch 62a with the top of slot 30. It is to be noted that, when catch 62a reaches the top of slot 30 in guide 20a, it is retained in that position by ratchet-like detent 32. Exactly the same situation exists with guide 20b, its slot 30, wing 54b and its catch 62b.

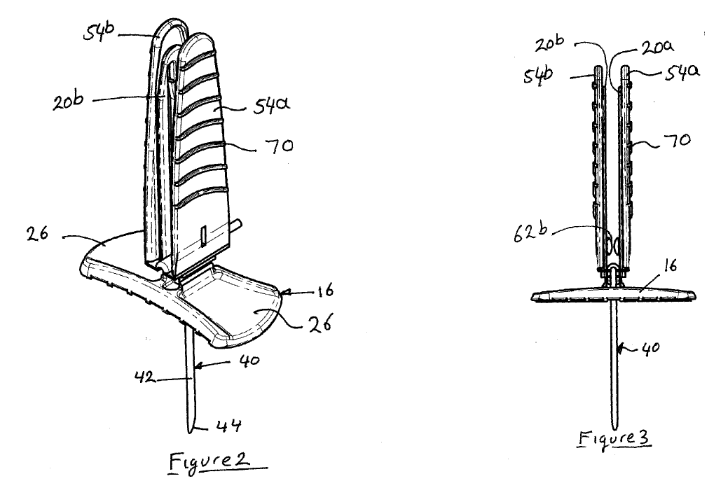

FIGS. 2 and 3 illustrate the appearance of Huber needle assembly 10 after assembly as described above. However, these figures do not show the needle sheath that would normally be fitted over the vertical portion 42 of Huber needle 40 before or after the above describe assembly process. With the sheath in place, needle assembly 10 is ready for packaging and sterilization.

To ready the needle assembly 10 for use (after unpacking), wings 54a and 54b are gripped firmly between the index finger and thumb of the right hand and the needle sheath (not shown) is removed using the left hand, so that the appearance of the assembly is as shown in FIG. 2. The assembly 10 is then positioned over the injection site and the vertical portion 42 of needle 40 is then inserted into the patient and the subcutaneous port. Considerable force can be exerted because of (i) the bulk and rigidity of the packed guide members and wings, (ii) the fact that wing members 54a and 54b are in abutment with foot 16 and (iii) foot 16 provides an abutment against which the finger and thumb can rest, should they slip downward on wings 54a and 54b—which, it will be noted, are provided with ribs 70 on their outer faces to reduce the likelihood of slip.

It is to be noted that needle assembly 10 is selected at the outset so that the length of the vertical portion 42 of needle 40 is appropriate for the depth to which the port has been implanted. This ensures that, after insertion, the bottom of foot 16 will be resting, directly or indirectly, on the patient's skin. [If desired at this stage, medical adhesive tape can be used to attach foot 16 to the patient's skin so that it is unlikely to move.]

At this stage, the user folds guide 20a and attached wing 54a, along with guide 20b and attached wing 54b, downwards and outwards to the horizontal position as shown in FIG. 5. Each guide/wing pair can then be taped down onto the patient's skin. A very flat and unobtrusive needle assembly thus results which is most unlikely to catch or snag the clothes of the user, those of the patient, associated bedclothes or other medical tubing or apparatus.

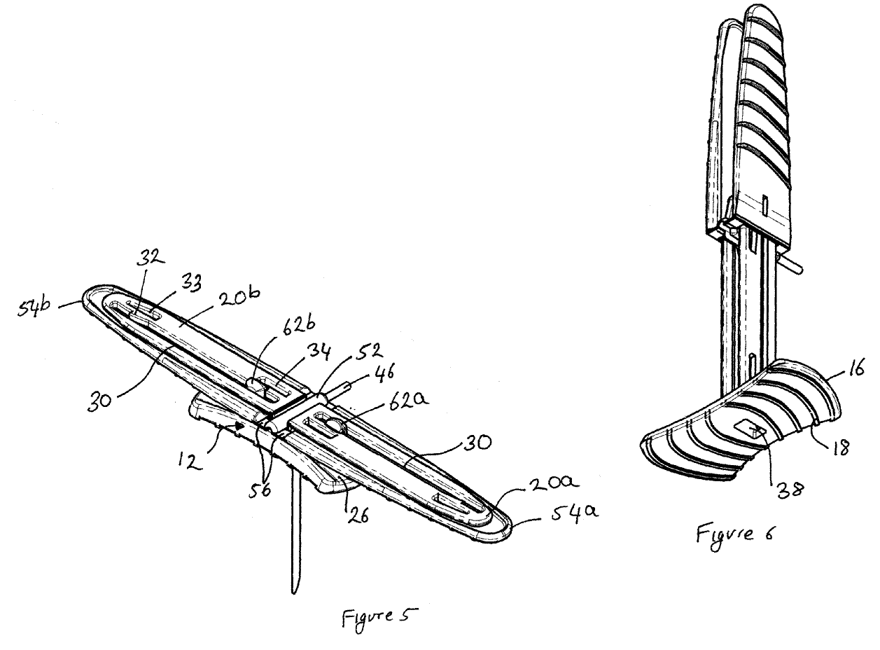

After sufficient fluid has been delivered (or removed), needle assembly 10 can be removed as follows. Any tape is removed, the guide/wing pairs are folded back to their vertical positions while making sure that foot 16 of base sub-assembly 12 is not bumped or moved relative to the patient. If thought prudent, foot 16 can be held in position with one hand while the guide/wing pairs are restored to vertical. Then, wings 54a and 54b are grasped between the thumb and index finger of one hand (usually the right) while foot 16 is held in position on the patient with the other hand and the wings are pulled up and away from foot 16. This is possible because wings 54a and 54b can slide on guide members 20a and 20b by virtue of the sliding engagement of catches 62a and 62b on wings 54a and 54b with respective slots 30 in guide members 20a and 20b. Raising wings 54a and 54b will, of course, also raise the hub 52 and Huber needle 40 until catches 62a and 62b are forced past the ratchet-like detents 32 to lock the wings against return. The resultant position is shown in FIGS. 6 and 7, wherein it can be seen that the vertical portion 42 of needle 40 is fully withdrawn upwards between guide members 20a and 20b and the needle assembly 10 can be simply lifted from the skin of the patient and placed in a suitable disposal container. At no time has the point of the used needle been exposed and, because foot 16 stays in contact with the patient at all times during removal of the needle, there is no chance of rebound needle-stick.

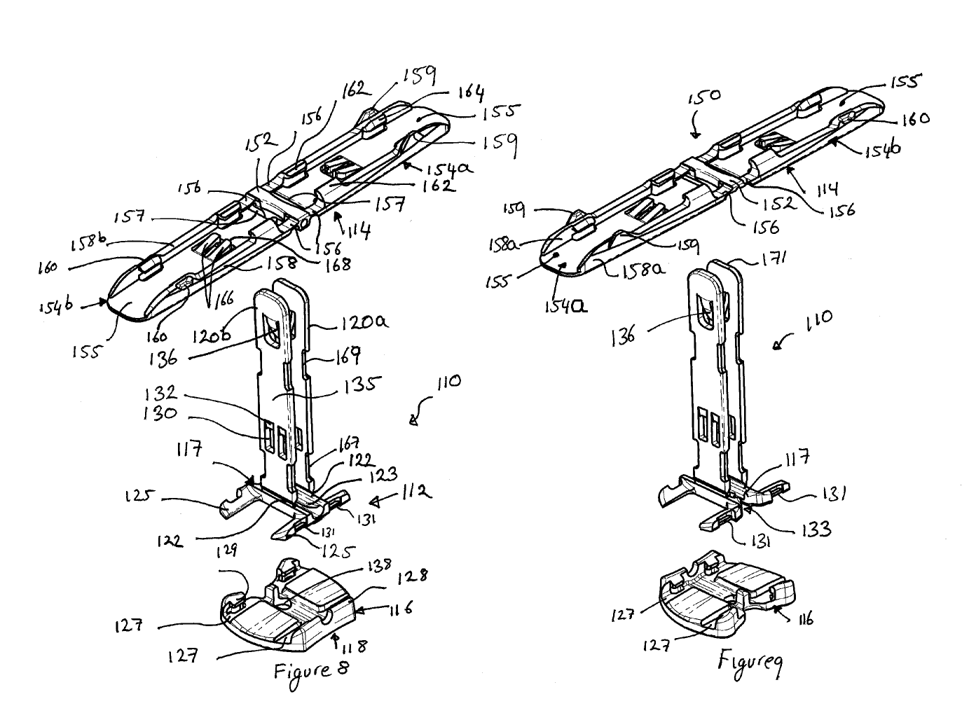

The Huber needle assembly of a second example, shown in FIGS. 8 to 16, is generally indicated at 110. The assembly 110 is comprised of base and needle sub-assemblies at 112 and 114.

The base sub-assembly 112 is injection-molded in two pieces from a plastic such as PVC or polypropylene. A first piece is an elongate foot 116 with a ribbed or patterned bottom face 118 (that is adapted to rest on the patient's skin above the injection site). The second piece 117 has a pair of opposed strip-like guide members 120a and 120b that are each attached by a narrow integral strip-hinge 122 to a central hub 123.

This hub 123 has legs 125 that are snap-fitted into grooves 127 in foot 116. Clips 129 on foot 116 engage tabs 131 on legs 125 to secure the two pieces 116 and 117 together. If desired the base sub-assembly 112 may be integrally formed in one piece.

Guide members 120a and 120b can be swung outwards and downwards about their respective hinges 122 to lie on or near the upper surface 126 of foot 116 which is channeled or grooved for the purpose, having raised sides 128.

Two parallel apertures or slots 130 are provided in each guide member near the hinge 122. The apertures are generally rectangular and extend lengthways along each guide member 120. The upper end 132 of each slot 130 is chamfered. In the example the slots 130 extend fully through the thickness of the guide members. This is not essential and, as an alternative, elongate depressions may be formed in the outer or lower surfaces 135 of the guide members 120.

A hole 138 is formed in the foot 116 between strip hinges 122 of guide members 120a and 120b through which the pointed portion of a Huber needle can extend. In this example the hole 138 is offset to one side but may be located centrally, as in the first example. The hub 123 has a groove 133 to one side through which the Huber needle may pass. If desired an aperture may be provided instead.

The guide members 120 each also include a tab 136 that extends upwards from the upper surface 137 of the guide member and with a free end 139 extending toward the respective hinge 122. The tab 136 may be deflected to lie in the plane of the guide member by application of suitable force but is resilient and will spring back to the extended position when released.

The needle sub-assembly 114 includes a bent hollow steel Huber needle 140 having a vertical portion 142, formed with a bent lower portion 143 terminating in point 144, and a horizontal butt portion 146 to the end of which a flexible tube (not shown) is attached for conveying fluid to or from the lumen of needle 140. Sub-assembly 114 also includes a one-piece plastic molding 150 having a central cylindrical hub 152 that is molded onto butt portion 146 of needle 140 and a pair of lateral outer wings 154a and 154b attached to hub 152 by integral strip-hinges 156. An elongate slot 157 is formed in the center of each strip-hinge 156 and, in fact, reduces the strip-hinge to a two peripheral or end portions. Hook-like catches 162 and 164 are formed on walls 158a and 158b so as to be upstanding from upper or inner faces 155 of wings 154a and 154b, respectively. The catches 162, 164 are sized so as to form a slot between themselves and the inner surfaces 155 into which the guide members 120 may slide.

Each outer wing has a pair of parallel tabs 166. These tabs 166 extend upwards out of the plane of the inner face 155 and extend with a free end 168 nearer the hinge 156.

The outer ends of the walls 158a on wing 154a have portions 159 that extend away from the inner face 155 more than the remainder of the walls 158. When the outer wings are squeezed together these portions 159 engage in corresponding recesses 160 in walls 158b of wing 154b before the remainder of the wings. This limits or prevents the squeezing action from jamming the inner guide members 120 and outer wings 154 together and stopping them from sliding relative to each other.

Base sub-assembly 112 and needle sub-assembly 114 are assembled by the following steps:
The needle sub-assembly 114 is positioned with the outer wings 154 horizontal. The base sub-assembly 112 is positioned with the guide members 120 vertical and generally parallel to each other;
The two sub-assemblies are moved toward one another so that vertical portion 142 of needle 140 is aligned over aperture 138 and the ends 171 of the guide members are aligned with the slots 157;
The upper ends 171 of guide members 120a and 120b are entered into respective slots 157 in hinges 156 of wings 154a and 154b by moving the outer wings downwards;
Continuing this downwards movement until the hub 152 contacts the tabs 136. Continued downwards movement deflects the tabs 136 outwards and once the hub has cleared the tabs 136 the tabs 136 spring inwards, so preventing the needle sub-assembly 114 being withdrawn upwards beyond the tabs 136,
Continuing this movement until needle point 144 enters and passes through hole 138 in foot 116, and hub 152 contacts hub 123 of the guide member component, and
Folding the guide members 120a, 120b to the horizontal position, as shown in FIG. 10. As the guide members 120a, 120b are folded to the horizontal position they snap-fit under the tabs 162, 164 on the outer wings 154.

Although the guide members 120a, 120b have recesses 167 and 169 that align with the tabs 162, 164, the tabs 162, 164 overlie the guide members 120 in this position and retain the guide members between the tabs and the inner surfaces 155. Recesses 167 and 169 are provided to make this snap fitting easier. Similarly the outer wings 154 are provided with apertures 173, 175 that allow the tabs 162, 164 to more easily bend to allow this snap-fitting.

A needle guard 177 may be located over the needle 140.

The guide members 120 and outer wings 154 may then be rotated to be substantially vertical. When vertical the outer wings 154 are relatively free to move vertically relative to the guide members 120, albeit restrained by tabs 166 and 136.

When the outer wings 154 are fully depressed vertically, the two tabs 166 extend into the apertures 130 in the guide members 120 and hold the outer sub assembly 114 in this position against accidental upwards movement during handling or preparation for use.

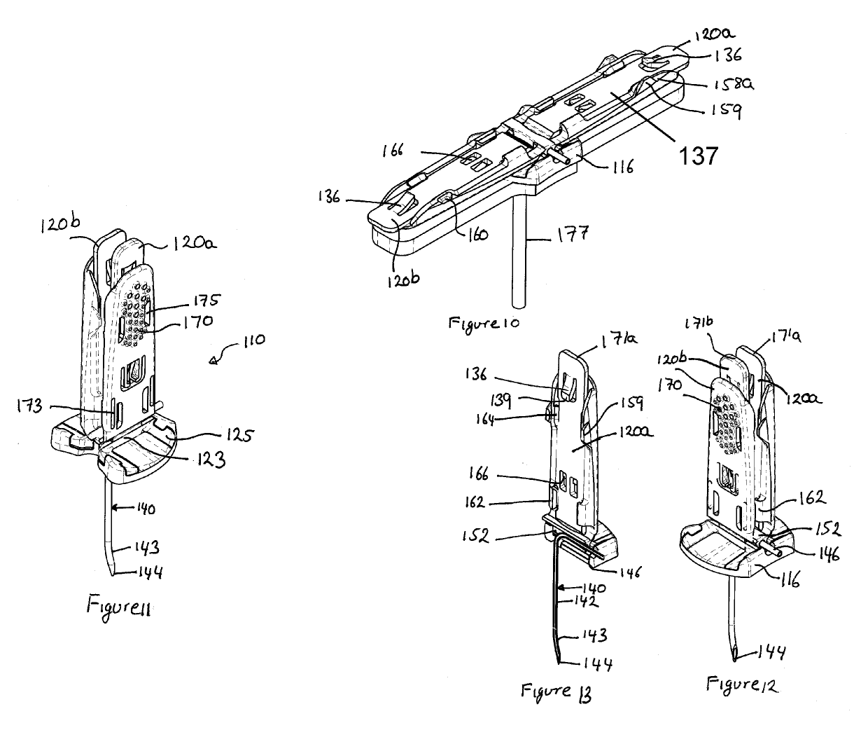

FIGS. 11 to 13 illustrate the appearance of Huber needle assembly 110 after assembly as described above. FIG. 11 does not show the needle sheath 177 that would normally be fitted over the vertical portion 142 of Huber needle 140 before or after the above described assembly process. With the sheath in place, needle assembly 110 is ready for packaging and sterilization.

To ready the needle assembly 110 for use (after unpacking), wings 154a and 154b are gripped firmly between the index finger and thumb of the right hand and the needle sheath 177 is removed using the left hand, so that the appearance of the assembly is as shown in FIG. 10. The assembly 110 is then positioned over the injection site and the vertical portion 142 of needle 140 is then inserted into the patient and the subcutaneous port. Considerable force can be exerted because of (i) the bulk and rigidity of the packed guide members and wings, (ii) the fact that wing members 154a and 154b are in abutment with foot 116 and (iii) foot 116 provides an abutment against which the finger and thumb can rest, should they slip downward on wings 154a and 154b—which, it will be noted, are provided with patterning 170 on their outer faces to reduce the likelihood of slip.

It is to be noted that needle assembly 110 is selected at the outset so that the length of the vertical portion 142 of needle 140 is appropriate for the depth to which the port has been implanted. This ensures that, after insertion, the bottom of foot 116 will be resting on padding, such as a layer of foam, sandwiched between the foot 116 and the patient's skin. [If desired at this stage, medical adhesive tape can be used to attach foot 116 to the patient's skin so that it is unlikely to move.]

At this stage, the user folds guide 120a and attached wing 154a, along with guide 120b and attached wing 154b, downwards and outwards to the horizontal position, similar to that shown in FIG. 10. Each guide/wing pair can then be taped down onto the patient's skin. A very flat and unobtrusive needle assembly thus results which is most unlikely to catch or snag the clothes of the user, those of the patient, associated bedclothes or other medical tubing or apparatus.

After sufficient fluid has been delivered (or removed), needle assembly 110 can be removed as follows. Any tape is removed, the guide/wing pairs are folded back to their vertical positions while making sure that foot 116 of base sub-assembly 112 is not bumped or moved relative to the patient. If thought prudent, foot 116 can be held in position with one hand while the guide/wing pairs are restored to vertical, as shown in FIGS. 11 and 12. Then, wings 154a and 154b are grasped between the thumb and index finger of one hand (usually the right) while foot 116 is held in position on the patient with the other hand and the wings are pulled up and away from foot 116. This is possible because wings 154a and 154b can slide relative to the guide members by virtue of the sliding engagement of the guide members 154 between the tabs 162 and 164.

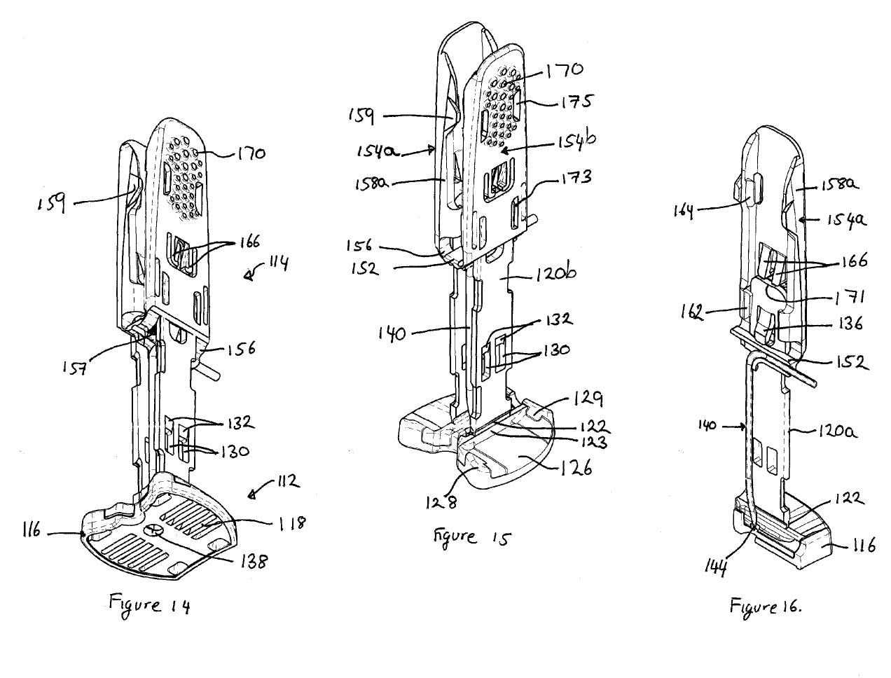

Raising wings 154a and 154b will, of course, also raise the hub 152 and Huber needle 140 until the tabs 166 are raised above the free ends of the guide members. At this position the tabs 166 spring inwards and extend over the free ends 171 of the guide members 120. The resultant position is shown in FIGS. 14 to 16.

In this position the point 144 of the needle 140 is withdrawn into the base 16 and is not exposed to the user. In this embodiment the vertical portion 142 of the needle lies to one side but between the guide members 120. Whilst the vertical portion 142 may be accessible to the user, the point 144 is not.

The locking of the tabs 166 over the free ends 171 of the guide members prevents the outer wings 154 and needle 140 from being lowered relative to the guide members and so any “jerking” as the needle is withdrawn from the patient cannot result in the needle extending out of the aperture 138.

The tabs 136 on the guide members 120 extend inwards and engage the hub 152 and so prevent further upwards movement of the outer wings and needle relative to the guide members and so prevent separation of the two sub assemblies, which would expose the needle 140. Thus once the needle sub-assembly 114 has been raised to this position it is locked in this position and cannot be depressed or raised further and the needle remains guarded between the guide members 120.

The entire needle assembly 110 can be simply lifted from the skin of the patient and placed in a suitable disposal container. At no time has the point of the used needle been exposed and, because foot 116 stays in contact with the patient or padding on the patient at all times during removal of the needle, there is no chance of rebound needle-stick.

It will be appreciated that the second example may be used with a straight needle and the first example may be used with a bent needle. Further, the second example may have the needle located centrally. The second example may have the needle point located above the aperture in the base and between the guide members, in a similar manner to the first example.

While two examples have been described and illustrated, and various modifications indicated, it will be appreciated that many changes to the example can be made without departing from the scope of the present invention as outlined above.

1. A needle assembly including:
a base sub-assembly having:
a foot portion having a generally horizontal base surface adapted to rest, directly or indirectly, on a patient's skin and having a passageway through which a needle may pass into the patient, and
at least one guide member connected to the foot portion and movable between a generally vertical position and a generally horizontal position, and
a needle sub-assembly, including a needle having a point and a hub formed on the needle, slidably mounted on the at least one guide member
wherein, when the at least one guide member is in the generally vertical position, the needle sub-assembly may slide along the at least one guide member, for insertion and/or withdrawal of the needle, between an extended position, in which the needle extends through the passageway and a retracted position in which the needle point is withdrawn away from the base surface
wherein at least part of the at least one guide member extends through at least one opening or slot in the needle sub assembly.

2. (canceled)

3. The needle assembly of claim 1 wherein the needle sub-assembly includes at least one finger grip attached to the hub for use in manipulating the needle point during insertion and withdrawal and wherein the at least one finger grip is movable between a generally vertical position in which it can be used to insert and/or remove the needle and a generally horizontal position in which it lies out of the way after the needle has been inserted.

4. The needle assembly of claim 3 wherein the at least one finger grip is hinged to the hub.

5. The needle assembly of claim 4 wherein said at least one opening or slot through which the at least one guide member extends is between the hub and the at least one finger grip.

6. The needle assembly of claim 5 wherein the needle sub-assembly includes two finger grips extending from either side of the hub.

7. The needle assembly of claim 5 wherein the at least one finger grip is mounted to the hub by two strip-like hinges, the two strip like hinges defining an opening or slot therebetween through which a corresponding guide member extends.

8. The needle assembly of claim 5 wherein the at least one guide member is hingedly mounted on the foot portion.

9. (canceled)

10. The needle assembly of claim 6 wherein two guide members extend between two finger grips.

11. The needle assembly of claim 10 wherein the hub is located between the two guide members.

12. The needle assembly of claim 5 wherein the at least one guide member, in the generally horizontal position, overlies at least part of the needle sub assembly and prevents or resists upwards movement of the needle sub-assembly toward the retracted position.

13. The needle assembly of claim 5 including at least one guide slot on one of the guide means and the needle sub assembly and at least one corresponding guide tab on the other of the guide means and the needle sub assembly that engages in the guide slot.

14. The needle assembly of claim 13 wherein the guide slot has a locking detent and/or a protrusion that, once the needle sub assembly has been moved to the retracted position, prevents movement of the guide tab and the needle sub assembly past the detent toward the extended position.

15. The needle assembly of claim 5 wherein the needle sub-assembly includes at least one first resilient leg that, once the needle sub assembly has been moved to the retracted position, overlies at least one free end of the at least one guide member and prevents or limits movement of the needle sub assembly toward the extended position.

16. The needle assembly of claim 5 wherein in the retracted position the point of the needle is located within the passageway.

17. The needle assembly of claim 5 wherein in the retracted position the point of the needle is at least partially shrouded by the at least one guide means.

18. The needle assembly of claim 5 wherein the at least one guide member acts as a needle guard before and/or after withdrawal of the needle, when the at least one guide member is substantially vertical.

19. The needle assembly of claim 5 including locking means between the needle sub-assembly and the base sub-assembly so that, after withdrawal to the retracted position, the needle point cannot be re-extended or re-exposed.

20. The needle assembly of claim 5 including retaining means to prevent the needle sub-assembly being separated from the base sub-assembly.

21. The needle assembly of claim 5 wherein, when assembled, the at least one guide member is movable between the horizontal and vertical positions only when the needle sub-assembly is in the extended position.

22. The needle assembly of claim 5 including at least one second resilient leg on the at least one guide member that engages a part of the needle sub-assembly to prevent the needle sub-assembly being separated from the base sub-assembly.

23. The needle assembly of claim 22 wherein the at least one second resilient leg engages the needle hub.

24. The needle assembly of claim 5 wherein, in the generally horizontal position, the at least one guide member extends away from the passageway after the needle has been inserted.

25. A needle assembly including:
a base sub-assembly having:
a foot portion having a generally horizontal base surface adapted to rest, directly or indirectly, on a patient's skin and having a passageway through which a needle may pass into the patient, and
two guide members connected to the foot portion with the passageway between the two guide members and movable between a generally vertical position and a generally horizontal position in which the two guide members extend in opposite directions away from the passageway, and
a needle sub-assembly slidably mounted on the two guide members, the needle sub-assembly including:
a needle having a point;
a hub formed on the needle;
two finger grips extending from either side of the hub for use in manipulating the needle point during insertion and withdrawal, each said finger grip mounted to the hub by two strip-like hinges, each pair of strip like hinges defining an opening or slot through which a respective one of said guide members extends such that the hub is located between the two guide members;
wherein,
when two guide members are in the generally vertical position, the needle sub-assembly may slide along the guide members, for insertion and/or withdrawal of the needle, between an extended position, in which the needle extends through the passageway and a retracted position in which the needle point is withdrawn away from the base surface; and
when needle sub-assembly is at the extended position and the guide members are in the generally horizontal position, the guide members overlay at least part of the needle sub assembly and prevent or resist upwards movement of the needle sub-assembly toward the retracted position;
one of each finger grip and respective guide member having at least one guide slot that engages the other of the finger grip and respective guide member, and
each finger grip including at least one first leg that, once the needle sub assembly has been moved to the retracted position, engages the respective guide member and prevents or limits movement of the needle sub assembly toward the extended position;
each guide member including a portion that engages a part of the needle sub-assembly to prevent the needle sub-assembly being separated from the base sub-assembly.
